(19) United States
(12) Patent Application Publication
(10) Pub. No.: US 2023/0201309 A1
(43) Pub. Date: Jun. 29, 2023

(54) COMPOSITIONS FOR THE TREATMENT OF A RESPIRATORY CONDITION

(71) Applicant: VECTURA LIMITED, Chippenham Wiltshire (GB)

(72) Inventor: Fergus MANFORD, Chippenham Wiltshire (GB)

(21) Appl. No.: 17/928,271

(22) PCT Filed: Mar. 26, 2021
(86) PCT No.: PCT/EP21/58041

§ 371 Date: Nov. 28, 2022

(30) Foreign Application Priority Data
Mar. 27, 2020 (EP) 20166525.4

(51) Int. Cl. A61K 38/21; A61K 31/4706; A61K 31/7052; A61P 31/14

(52) U.S. Cl. CPC A61K 38/215; A61K 38/212; A61K 31/4706; A61K 31/7052; A61P 31/14

(57) ABSTRACT

The present disclosure relates to the administration of a composition comprising an interferon, azithromycin and chloroquine for the treatment of respiratory conditions.

### BACKGROUND

Severe acute respiratory syndrome coronavirus 2 (SARS-CoV-2) is a positive-sense single-stranded RNA virus. This virus is the cause of the ongoing pandemic of coronavirus disease 2019 (COVID-19) that has been designated a Public Health Emergency of International Concern by the World Health Organization (WHO). The reproduction number (Ro) of SARS-CoV-2 has been calculated to be between 1.4 and 3.9; meaning that each infection from the virus is expected to result in 1.4 to 3.9 new infections (assuming nobody is immune and no preventive measures, such as social distancing, are implemented).

SARS-CoV-2 is a strain of the species severe acute respiratory syndrome-related coronavirus. The likely source of SARS-CoV-2 is a bat coronavirus. The pangolin is thought to be an intermediate animal reservoir involved in the introduction to humans.

Transmission of SARS-CoV-2 occurs primarily via respiratory droplets and from indirect contact via contaminated surfaces. Viral RNA has also been found in stool samples from infected people. Initial studies suggest that the virus may remain viable on untreated plastic and steel surfaces for up to 72 hours. The virus may remain viable on cardboard for less than 24 hours. On copper, the virus does not remain viable for more than four hours.

It remains to be determined whether humans are infectious during the incubation period; there are conflicting accounts in this regard. On 1 Feb. 2020, the WHO stated that transmission from infected but asymptomatic cases is unlikely to be a major driver of transmission. In contrast, an epidemiological study of the outbreak in China suggests that pre-symptomatic viral shedding may be common and that subclinical infections may have been the source of a majority of the infections reported in China.

Structurally, each SARS-CoV-2 virion is approximately 50-200 nanometres in diameter. The SARS-CoV-2 virion has four structural proteins, known as the S (spike), E (envelope), M (membrane), and N (nucleocapsid) proteins. The spike protein is responsible for allowing the virus to attach to the membrane of a host cell. The spike protein of the SARS-CoV-2 virion has sufficient affinity to the angiotensin converting enzyme 2 (ACE2) receptors of human cells. Independent research groups in China and the United States have experimentally demonstrated that ACE2 could act as the receptor for SARS-CoV-2. ACE2 is a type 1 integral membrane glycoprotein that is expressed and active in most tissues. The highest expression of ACE2 is observed in the kidney, the endothelium, the lungs and the heart.

To gain entry into the target cell, spike protein priming for the SARS-CoV-2 is required. After a SARS-CoV-2 virion attaches to the cell, a protease exposes a fusion peptide by cleaving the spike protein. Viral RNA is then released into the cell.

On 26 Mar. 2020 the British Medical Journal published an article summarising the most promising treatments for COVID-19. The WHO has now launched the SOLIDARITY TRIAL to investigate four potential treatments: remdesivir, chloroquine/hydroxychloroquine; lopinavir and ritonavir; and lopinavir and ritonavir plus interferon-β.

There is currently no specific treatment for coronavirus; hospital treatment aims to relieve the symptoms whilst the body's immune system combats the virus.

Chronically ill COVID-19 patients require several days of oxygen therapy, invasive mechanical ventilation and intensive care from healthcare workers; many of whom still do not survive.

There is an urgent need for an effective treatment that is able to both treat symptomatic patients and simultaneously reduce the respiratory viral loads prior to hospital admission.

Whilst global healthcare systems continues to roll out numerous SARS-CoV-2 vaccines, these therapies are, by their nature, highly specific. Should the SARS-CoV-2 mutate there is the distinct possibility that existing vaccines might be rendered ineffective against such mutations.

The world has witnessed the amazing speed with which vaccines can be produced but they still take several months to manufacture. During which time many hundreds of thousands of people may die as a result of infection with the new strain.

A therapy with broad viral efficacy that is capable of reducing the respiratory viral loads prior to hospital admission would dramatically reduce the severity of disease whilst these patients remain in hospital and have great utility in improving the long-term prognosis of these patients.

There remains an urgent need for the broad and effective treatment, amelioration and prevention of SARS-CoV-2 infection in humans.

### SUMMARY OF INVENTION

We disclose an improved composition comprising an interferon, azithromycin and chloroquine for use in a method of prevention or treatment of a respiratory condition. In particular, the composition comprises interferon-β 1a, azithromycin and hydroxychloroquine for the treatment of the disease COVID-19.

Unlike current therapies that use single actives such as chloroquine or interferon-β 1a or combination therapies such as azithromycin and hydroxychloroquine, the combination of interferon-β 1a, azithromycin and hydroxychloroquine achieves several outcomes: it reduces viral loads in patients infected with the virus SARS-CoV-2 thereby reducing community transmission, it reduces the respiratory viral loads in patients prior to hospital admission thereby presenting less of a risk to healthcare workers and it reduces the duration of stay in hospital.

The constituent active pharmaceutical ingredients in this triple combination composition are readily available and cost effective.

### DETAILED DESCRIPTION OF THE INVENTION

In a preferred embodiment of the invention a composition comprising interferon, azithromycin and chloroquine for use in a method of treating COVID-19 is disclosed, wherein the method comprises administering a composition comprising interferon, azithromycin and chloroquine to a patient infected with SARS-CoV-2.

In a preferred embodiment a composition comprising interferon, azithromycin and chloroquine for use in a method of prevention or treatment of a respiratory condition is disclosed.

In a preferred embodiment the respiratory condition is a severe acute respiratory condition.

In a preferred embodiment the respiratory condition or severe acute respiratory condition is coronavirus disease 2019 (COVID-19).

In a preferred embodiment the respiratory condition or severe acute respiratory condition is severe acute respiratory syndrome (SARS).

In a preferred embodiment the respiratory condition or severe acute respiratory condition is Middle East respiratory syndrome (MERS).

In a preferred embodiment the composition is a dry powder formulation.

In a preferred embodiment the composition is a pressurised metered dose inhaler formulation delivered to the patient by a pressurised metered dose inhaler.

In a preferred embodiment the composition is a nebulised formulation.

In a preferred embodiment the composition is a combination of a dry powder formulation and a nebulised formulation, delivered by a nebuliser inhaler or atomisation device.

In a preferred embodiment the composition is a combination of a dry powder formulation and a nebulised formulation, delivered by a nebuliser inhaler and dry powder inhaler.

In a preferred embodiment the composition is a combination of a dry powder, pressurised metered dose inhaler formulation and a nebulised formulation.

In a preferred embodiment the composition is a combination of a dry powder, pressurised metered dose inhaler formulation and a nebulised formulation delivered by a dry powder inhaler, pressurised metered dose inhaler and nebuliser inhaler.

In a preferred embodiment the composition is made by a process wherein a pharmaceutically active ingredient (API) is co-milled with a pharmaceutical additive.

In a preferred embodiment the composition comprises a pharmaceutical additive which is selected from a metal stearate, sodium lauryl sulphate, sodium stearyl fumarate, sodium stearyl lactylate, preferably calcium stearate, lithium stearate, magnesium stearate, sodium stearate, zinc stearate, stearyl alcohol or sodium benzoate. An especially preferred additive material comprises magnesium stearate.

In a preferred embodiment the composition comprises a pharmaceutical excipient which is selected from lactose, mannitol, glucose, trehalose, cellobiose, sorbitol or maltitol, more preferably anhydrous lactose, more preferably alpha-lactose monohydrate.

In a preferred embodiment the composition is for nasal administration.

In a preferred embodiment the composition is for pulmonary administration.

In a preferred embodiment the composition is for oropharyngeal administration.

In a preferred embodiment the composition is for nasal and pulmonary administration.

In a preferred embodiment the composition is for nasal, pulmonary and oropharyngeal administration.

In a preferred embodiment the interferon, azithromycin and chloroquine is administered to a patient simultaneously or sequentially.

In a preferred embodiment the composition has a bimodal particle size distribution as determined by laser diffraction.

In a preferred embodiment the bimodal composition has a lowest local maxima having a particle size of from 0.5 μm to 10 μm and a highest local maxima having a particle size from 30 μm to 120 μm as determined by laser diffraction.

In a preferred embodiment the composition has a trimodal particle size distribution as determined by laser diffraction.

In a preferred embodiment the trimodal composition has a lowest local maxima having a particle size of from 0.5 μm to 10 μm and a highest local maxima having a particle size from 30 μm to 120 μm as determined by laser diffraction.

In a preferred embodiment the trimodal composition has an intermediate local maxima having a particle size between 10 μm and 30 μm as determined by laser diffraction.

In a preferred embodiment the chloroquine is chloroquine phosphate.

In a preferred embodiment the chloroquine is hydroxychloroquine, preferably hydroxychloroquine sulfate.

In a preferred embodiment the interferon is interferon-α.

In a preferred embodiment the interferon is interferon-β.

In a preferred embodiment the interferon is interferon-β 1a.

In a preferred embodiment the interferon is interferon-β 1b.

In a preferred embodiment the interferon is interferon-ε.

In a preferred embodiment the interferon is interferon-κ.

In a preferred embodiment the interferon is interferon-ω.

In a preferred embodiment the interferon is a combination of interferon-α and interferon-β, optionally interferon-β 1a or interferon-β 1b or combination of interferon-β 1a and interferon-β 1b.

In a preferred embodiment a pharmaceutically active ingredient is formulated as a composite active particle comprising a taste masking agent.

In a preferred embodiment the chloroquine is formulated as a composite active particle comprising a taste masking agent.

In a preferred embodiment the taste masking agent is magnesium stearate.

In a preferred embodiment the hydroxychloroquine is formulated as a composite active particle comprising a taste masking agent, preferably wherein the taste masking agent is magnesium stearate.

In a preferred embodiment the chloroquine or acceptable salt thereof is present in a composition to be administered to a patient in an amount of from 10 mg to 1000 mg, preferably from 20 mg to 900 mg, preferably from 30 mg to 800 mg, preferably from 40 mg to 700 mg, preferably from 50 mg to 600 mg, preferably from 60 mg to 500 mg, preferably from 70 mg to 400 mg, preferably from 80 mg to 300 mg, preferably from 90 mg to 200 mg.

In a preferred embodiment the chloroquine phosphate is present in a composition to be administered to a patient in an amount of from 10 mg to 1000 mg, preferably from 20 mg to 900 mg, preferably from 30 mg to 800 mg, preferably from 40 mg to 700 mg, preferably from 50 mg to 600 mg, preferably from 60 mg to 500 mg, preferably from 70 mg to 400 mg, preferably from 80 mg to 300 mg, preferably from 90 mg to 200 mg.

In a preferred embodiment the hydroxychloroquine or acceptable salt thereof is present in a composition to be administered to a patient in an amount of from 10 mg to 1000 mg, preferably from 20 mg to 900 mg, preferably from 30 mg to 800 mg, preferably from 40 mg to 700 mg, preferably from 50 mg to 600 mg, preferably from 60 mg to 500 mg, preferably from 70 mg to 400 mg, preferably from 80 mg to 300 mg, preferably from 90 mg to 200 mg.

In a preferred embodiment the hydroxychloroquine sulfate is present in a composition to be administered to a patient in an amount of from 10 mg to 1000 mg, preferably from 20 mg to 900 mg, preferably from 30 mg to 800 mg, preferably from 40 mg to 700 mg, preferably from 50 mg to 600 mg, preferably from 60 mg to 500 mg, preferably from 70 mg to 400 mg, preferably from 80 mg to 300 mg, preferably from 90 mg to 200 mg.

In a preferred embodiment the interferon is administered by intravenous injection.

In a preferred embodiment the interferon is administered by liquid aerosol to the patient.

In a preferred embodiment the interferon is administered by liquid aerosol to the patient for nasal administration.

In a preferred embodiment the interferon is administered by liquid aerosol to the patient for pulmonary administration.

In a preferred embodiment the interferon is administered by liquid aerosol to the patient for oropharyngeal administration.

In a preferred embodiment the interferon is administered by liquid aerosol to the patient for nasal and pulmonary administration.

In a preferred embodiment the interferon is administered by liquid aerosol to the patient for nasal, pulmonary and oropharyngeal administration.

In a preferred embodiment the azithromycin is administered by liquid aerosol to the patient for nasal administration.

In a preferred embodiment the azithromycin is administered by liquid aerosol to the patient for pulmonary administration.

In a preferred embodiment the azithromycin is administered by liquid aerosol to the patient for oropharyngeal administration.

In a preferred embodiment the azithromycin is administered by liquid aerosol to the patient for nasal and pulmonary administration.

In a preferred embodiment the azithromycin is administered by liquid aerosol to the patient for nasal, pulmonary and oropharyngeal administration.

In a preferred embodiment the chloroquine is administered by liquid aerosol to the patient for nasal administration.

In a preferred embodiment the chloroquine is administered by liquid aerosol to the patient for pulmonary administration.

In a preferred embodiment the chloroquine is administered by liquid aerosol to the patient for oropharyngeal administration.

In a preferred embodiment the chloroquine is administered by liquid aerosol to the patient for nasal and pulmonary administration.

In a preferred embodiment the chloroquine is administered by liquid aerosol to the patient for nasal, pulmonary and oropharyngeal administration.

In a preferred embodiment the interferon is present in an amount of from 50 μg to 500 μg, preferably from 60 μg to 450 μg, preferably from 70 μg to 400 μg, preferably from 80 μg to 350 μg, preferably from 90 μg to 300 μg, preferably from 100 μg to 250 μg.

In a preferred embodiment the interferon is present in an amount of from 1 ng to 500 ng, preferably from 2 ng to 475 ng, preferably from 3 ng to 450 ng, preferably from 4 ng to 425 ng, preferably from 5 ng to 400 ng, preferably from 6 ng to 375 ng, preferably from 7 ng to 350 ng, preferably from 8 ng to 325 ng, preferably from 9 ng to 300 ng, preferably from 10 ng to 275 ng, preferably from 11 ng to 250 ng, preferably from 12 ng to 225 ng, preferably from 13 ng to 200 ng, preferably from 14 ng to 175 ng, preferably from 15 ng to 150 ng.

In a preferred embodiment the interferon-β 1a. is present in an amount of from 1 ng to 500 ng, preferably from 2 ng to 475 ng, preferably from 3 ng to 450 ng, preferably from 4 ng to 425 ng, preferably from 5 ng to 400 ng, preferably from 6 ng to 375 ng, preferably from 7 ng to 350 ng, preferably from 8 ng to 325 ng, preferably from 9 ng to 300 ng, preferably from 10 ng to 275 ng, preferably from 11 ng to 250 ng, preferably from 12 ng to 225 ng, preferably from 13 ng to 200 ng, preferably from 14 ng to 175 ng, preferably from 15 ng to 150 ng.

In a preferred embodiment the azithromycin is present in an amount of from 100 mg to 1000 mg, preferably from 200 mg to 900 mg, preferably from 300 mg to 800 mg, preferably from 400 mg to 700 mg, preferably from 450 mg to 650 mg, preferably from 500 mg to 550 mg.

In a preferred embodiment the composition is administered to a patient once daily.

In a preferred embodiment the composition is administered to a patient twice daily.

In a preferred embodiment the composition is administered to a patient pro re nata (PRN).

In a preferred embodiment the composition is administered using a pulse dosing regimen.

In a preferred embodiment the interferon is administered via a separate route of administration to the azithromycin and chloroquine.

In a preferred embodiment the interferon is administered via same route of administration to the azithromycin and hydroxychloroquine, preferably wherein the interferon, azithromycin and hydroxychloroquine are administered via intravenous injection.

In a preferred embodiment the interferon is administered in combination with the azithromycin and chloroquine.

In a preferred embodiment a pharmaceutical kit is disclosed, comprising a composition comprising interferon, azithromycin and chloroquine, in combined or separate unit dosage forms, said forms being suitable for simultaneous or sequential administration of the actives in effective amounts, optionally for administration of the three actives with one or more respiratory devices.

In a preferred embodiment an inhaler is disclosed, comprising a composition comprising interferon, azithromycin and chloroquine, in combined or separate unit dosage forms, said forms being suitable for simultaneous or sequential administration of the actives in effective amounts, optionally for administration of the three actives with one or more inhaler devices.

In a preferred embodiment a composition is disclosed comprising interferon-β, azithromycin and chloroquine for use in the treatment of respiratory condition, wherein said treatment is by aerosol delivery of said medicament to the respiratory tract.

In a preferred embodiment a composition is disclosed comprising interferon, azithromycin and chloroquine for use in a method of treating COVID-19, wherein the method comprises administering the composition to a patient infected with coronavirus SARS-CoV-2.

In a preferred embodiment a composition is disclosed comprising interferon-β, azithromycin and hydroxychloroquine for use in the prevention or treatment of the disease COVID-19 in patients infected with the coronavirus SARS-CoV-2.

In a preferred embodiment a composition is disclosed comprising interferon-β, azithromycin and hydroxychloroquine for use in the prevention of coronavirus SARS-CoV-2 viral load within a patient.

In a preferred embodiment a composition is disclosed comprising interferon-β3, azithromycin and hydroxychloroquine for use in the reduction of coronavirus SARS-CoV-2 viral load within a patient.

In a preferred embodiment a therapeutic composition is disclosed, for treating, ameliorating or preventing coronavirus SARS-CoV-2 viral load within a patient.

In a preferred embodiment, the efficacy of the composition is established by measuring respiratory viral load, preferably determined by real-time reverse transcription-PCR.

In a preferred embodiment, 50% of original viral clearance is achieved within two days as determined by real-time reverse transcription-PCR of SARS-CoV-2 RNA.

In a preferred embodiment, 75% of original viral clearance is achieved within three days as determined by real-time reverse transcription-PCR of SARS-CoV-2 RNA.

In a preferred embodiment, 100% of original viral clearance is achieved before five days as determined by real-time reverse transcription-PCR of SARS-CoV-2 RNA.

### FIGURES

FIG. 1 is a summary showing the percentage of patients with PCR-positive nasopharyngeal samples from inclusion to day 10 post-inclusion in COVID-19 patients treated with either inhaled chloroquine only (Group 2), inhaled interferon-β 1a (Group 3), inhaled hydroxychloroquine and azithromycin (Group 4) or inhaled interferon-β 1a, hydroxychloroquine and azithromycin (Group 5), compared with COVID-19 control patients (Group 1).

### CLINICAL AND IN VITRO EVIDENCE PUBLISHED AFTER THE PRIORITY DATE OF THE APPLICATION

A study by Catteau et al. published on 24 Aug. 2020 in the International Journal of Antimicrobial Agents reported on a nationwide observational study of 8,075 participants and found there to be an effect of low-dose hydroxychloroquine therapy on mortality in hospitalised patients with COVID-19. Rather than use the extremely high doses of the RECOVERY TRIAL and SOLIDARITY TRIAL, this Belgium study administered 400 mg hydroxychloroquine sulphate in monotherapy and supportive care (hydroxychloroquine sulphate group) statim followed by 400 mg on Day 1, followed by 200 mg twice a day from Days 2 to 5, i.e. a total dose of 2400 mg. These patients were compared with a control group treated with supportive care only (no-hydroxychloroquine sulphate group). Of the 8,075 patients, 4,542 received hydroxychloroquine sulphate in monotherapy and 3,533 were in the no-hydroxychloroquine sulphate group. Hospital mortality was reported in 804/4,542 patients (17.7%) (hydroxychloroquine sulphate group) compared with 957/3,533 patients (27.1%) (no-hydroxychloroquine sulphate group). Using multivariate analysis, the authors report that mortality was lower in the hydroxychloroquine sulphate group compared with the no-hydroxychloroquine sulphate group [adjusted hazard ratio (aHR)=0.684, 95% confidence interval (CI) 0.617-0.758]. In other words, 1000 patients would survive in the hydroxychloroquine sulphate group, compared to only 684 surviving in the no-hydroxychloroquine sulphate group. Catteau et al. concluded that compared with supportive care only, low-dose hydroxychloroquine sulphate monotherapy was independently associated with lower mortality in hospitalised patients with COVID-19 diagnosed and treated early or later after symptom onset.

On 25 Apr. 2020, in the journal Microbial Pathogenesis, Andreani et al. reported that following in vitro testing of combined hydroxychloroquine and azithromycin on SARSCoV-2 showed a synergistic effect. In particular, the authors demonstrated that the combination of hydroxychloroquine and azithromycin has a synergistic effect in vitro on SARS-CoV-2 at concentrations compatible with that obtained in the human lung.

Finally, on 12 Nov. 2020, in the journal The Lancet Respiratory Medicine, Monk et al. reported the safety and efficacy of inhaled nebulised interferon beta-1a (SNG001) for treatment of SARS-CoV-2 infection during a phase 2 randomised, double-blind, placebo-controlled trial. The authors concluded that patients who received interferon-β 1a had greater odds of improvement and recovered more rapidly from SARS-CoV-2 infection than patients who received placebo only. Worth noting is that three patients died during the study; all deaths occurred in patients in the placebo group.

### Example

Fifty patients, aged between 10-90 yrs, presenting with positive PCR documented SARS-CoV-2 carriage are included in this study and randomly assigned to the following groups.

Group 1: Ten patients are untreated patients.

Group 2: Ten patients are treated with inhaled chloroquine.

Group 3: Ten patients are treated with inhaled interferon-β 1a.

Group 4: Ten patients are treated with inhaled hydroxychloroquine and azithromycin.

Group 5: Ten patients are treated with inhaled interferon-β 1a, hydroxychloroquine and azithromycin.

Group 1: Untreated Patients

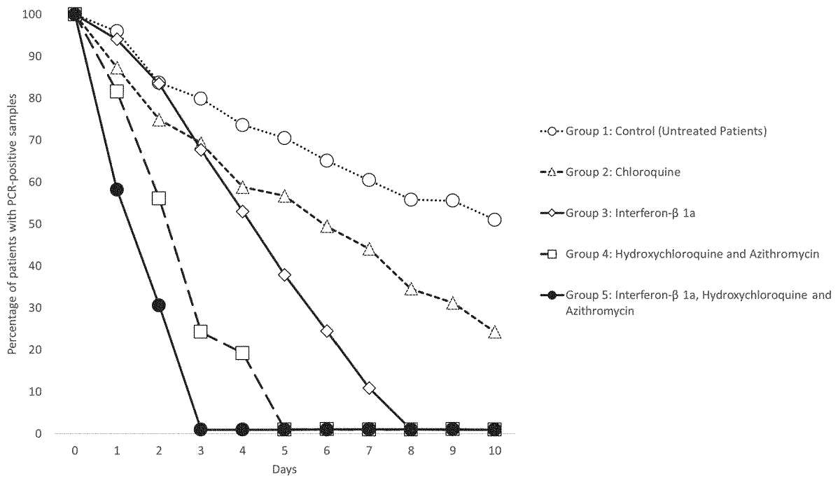

Viral clearance is determined each day by real-time reverse transcription-PCR of SARS-CoV-2 RNA from nasopharyngeal samples and the results reported in FIG. 1.

Group 2 Formulation: Inhaled Chloroquine Only

Unmicronised chloroquine (15 g, D₁₀>20 μm, D₅₀>100 μm, D₉₀>200 μm is determined by Malvern Mastersizer 3000 wet analysis method) and is pre-stirred in a glass beaker using a metal spatula for 30 seconds before micronization in an AS-50 spiral jet mill (Inlet pressure=5 Bar, Grinding Pressure=3 Bar, Averaged Feed Rate=2

Micronised chloroquine is combined with lactose carrier to create a uniform blend. A portion of the blend is then filled into a capsule. The capsule is fitted into an inhaler and a dose of chloroquine is administered to the patient by an inhalation manoeuvre. The dosing is repeated as required.

Viral clearance is determined each day by real-time reverse transcription-PCR of SARS-CoV-2 RNA from nasopharyngeal samples and the results reported in FIG. 1.

Group 3 Formulation: Inhaled Interferon-β 1a Only

Unmicronised interferon-β 1a (15 g, D₁₀>20 μm, D₅₀>100 μm, D₉₀>200 μm is determined by Malvern Mastersizer 3000 wet analysis method) and is pre-stirred in a glass beaker using a metal spatula for 30 seconds before micronization in an AS-50 spiral jet mill (Inlet pressure=5 Bar, Grinding Pressure=3 Bar, Averaged Feed Rate=2

Micronised interferon-β 1a is combined with lactose carrier to create a uniform blend. A portion of the blend is then filled into a capsule. The capsule is fitted into an inhaler and a dose of interferon-β 1a is administered to the patient by an inhalation manoeuvre. The dosing is repeated as required.

Viral clearance is determined each day by real-time reverse transcription-PCR of SARS-CoV-2 RNA from nasopharyngeal samples and the results reported in FIG. 1.

Group 4 Formulation: Inhaled Hydroxychloroquine and Azithromycin

Unmicronised hydroxychloroquine (15 g, D₁₀>20 μm, D₅₀>100 μm, D₉₀>200 μm is determined by Malvern Mastersizer 3000 wet analysis method) and is pre-stirred in a glass beaker using a metal spatula for 30 seconds before micronization in an AS-50 spiral jet mill (Inlet pressure=5 Bar, Grinding Pressure=3 Bar, Averaged Feed Rate=2 g/min).

Micronised Hydroxychloroquine is Combined with Lactose Carrier.

Unmicronised azithromycin (15 g, D₁₀>20 μm, D₅₀>100 μm, D₉₀>200 μm is determined by Malvern Mastersizer 3000 wet analysis method) and is pre-stirred in a glass beaker using a metal spatula for 30 seconds before micronization in an AS-50 spiral jet mill (Inlet pressure=5 Bar, Grinding Pressure=3 Bar, Averaged Feed Rate=2

Micronised azithromycin is combined with lactose carrier.

The two blends are combined to create a uniform blend. A portion of the combined blend is then filled into a capsule. The capsule is fitted into an inhaler and a dose of hydroxychloroquine and azithromycin is administered to the patient by an inhalation manoeuvre. The dosing is repeated as required.

Viral clearance is determined each day by real-time reverse transcription-PCR of SARS-CoV-2 RNA from nasopharyngeal samples and the results reported in FIG. 1.

Group 5 Formulation: Inhaled Hydroxychloroquine, Azithromycin and Interferon-β 1a

Unmicronised hydroxychloroquine sulfate (15 g, D₁₀>20 μm, D₅₀>100 μm, D₉₀>200 μm determined by Malvern Mastersizer 3000 wet analysis method) is pre-stirred in a glass beaker using a metal spatula for 30 seconds before micronization in an AS-50 spiral jet mill (Inlet pressure=5 Bar, Grinding Pressure=3 Bar, Averaged Feed Rate=2 g/min).

Micronised Hydroxychloroquine Sulfate is Combined with Lactose Carrier.

Unmicronised azithromycin (15 g, D₁₀>20 μm, D₅₀>100 μm, D₉₀>200 μm determined by Malvern Mastersizer 3000 wet analysis method) is pre-stirred in a glass beaker using a metal spatula for 30 seconds before micronization in an AS-50 spiral jet mill (Inlet pressure=5 Bar, Grinding Pressure=3 Bar, Averaged Feed Rate=2 g/min).

Micronised Azithromycin is Combined with Lactose Carrier.

The two blends are combined to create a uniform blend. A portion of the combined blend is then filled into a capsule to create a nominal dose. The capsule is fitted into an inhaler and a dose of hydroxychloroquine sulfate (200 mg per nominal dose) and azithromycin (500 mg per nominal dose) is administered to the patient by an inhalation manoeuvre. The dosing is repeated as required.

The interferon-β 1a (250 μg per nominal dose) is in solution to be administered sequentially by nebuliser. The dosing is repeated as required.

Alternatively, the interferon-β 1a (250 μg per nominal dose) is administered simultaneously as a dry powder with the hydroxychloroquine sulfate and azithromycin. The dosing is repeated as required.

The formulations are assessed by laser diffraction prior to blending with carrier and the APIs are found to exhibit a trimodal distribution having a lowest local maxima with a particle size of from 0.5 μm to 10 μm, an intermediate local maxima having a particle size between 10 μm and 30 μm and a highest local maxima with a particle size from 30 μm to 120 μm. The three distributions being eminently suitable for simultaneously targeting the pulmonary, nasal and oropharyngeal epithelium.

Viral clearance is determined each day by real-time reverse transcription-PCR of SARS-CoV-2 RNA from nasopharyngeal samples and the results reported in FIG. 1.

FIG. 1 demonstrates that inhaled hydroxychloroquine, azithromycin and interferon-β 1a (Group 5) yields the quickest reduction in viral loading compared with other formulations (Group 1-4).

1. A method for preventing or treating a respiratory condition, comprising:
administering to a patient a composition comprising an interferon, azithromycin and chloroquine.

2. The method of claim 1, wherein the respiratory condition is a severe acute respiratory condition selected from the group consisting of of coronavirus disease 2019 (COVID-19), severe acute respiratory syndrome (SARS) and Middle East respiratory syndrome (MERS).

3. The method of claim 1, wherein the respiratory condition is a mild respiratory condition selected from the group consisting of coronavirus disease 2019 (COVID-19), severe acute respiratory syndrome (SARS) and Middle East respiratory syndrome (MERS).

4. The method of claim 1, wherein the composition is at least one of a dry powder formulation, a pressurised metered dose inhaler formulation and a nebulised formulation.

5. The method of claim 1, wherein the composition is administered by at least one of pulmonary administration, nasal administration and oropharyngeal administration.

6. The method of claim 1, wherein the interferon, azithromycin and chloroquine are administered to the patient simultaneously or sequentially.

7. The method of claim 4, wherein the composition has a bimodal particle size distribution as determined by laser diffraction.

8. The method of claim 4, wherein the composition has a trimodal particle size distribution as determined by laser diffraction.

9. The method of claim 7, wherein a lowest local maxima of the composition has a particle size of from 0.5 μm to 10 μm and a highest local maxima of the composition has a particle size from 30 μm to 120 μm, as determined by laser diffraction.

10. The method of claim 8, wherein an intermediate local maxima of the composition has a particle size between 10 μm and 30 μm, as determined by laser diffraction.

11. The method of claim 1, wherein the chloroquine is at least one of chloroquine phosphate or hydroxychloroquine or an acceptable salt thereof.

12. The method of claim 1, wherein the interferon is at least one of interferon-α and interferon β.

13. The method of claim 1, wherein the interferon is present in an amount of from 50 μg to 500 μg.

14. The method of claim 1, wherein the azithromycin is present in an amount of from 100 mg to 1000 mg.

15. The method of claim 16, wherein either the chloroquine phosphate or hydroxychloroquine sulfate is present in an amount of from 10 mg to 1000 mg.

16. The method of claim 1, wherein the hydroxychloroquine is hydroxychloroquine sulfate.

17. The method of claim 1, wherein the interferon is interferon-β 1a.

18. The method of claim 1, wherein the interferon is interferon-β 1b.
